(19) United States
(12) Patent Application Publication
(10) Pub. No.: US 2024/0093592 A1
(43) Pub. Date: Mar. 21, 2024

(54) QUANTIFICATION OF PORE-FILLING DOLOMITE AND CALCITE CEMENT IN CARBONATE RESERVOIRS IN POST HYDROCARBON CHARGE STAGE

(71) Applicant: SAUDI ARABIAN OIL COMPANY, Dhahran (SA)

(72) Inventors: Abrar Alabbad, Al-Jish (SA); Wisam Kawai, Qatif (SA); Zainab Al Ibrahim, Safwa (SA)

(73) Assignee: SAUDI ARABIAN OIL COMPANY, Dhahran (SA)

(21) Appl. No.: 17/933,927

(22) Filed: Sep. 21, 2022

(51) Int. Cl. E21B 47/005; E21B 49/02; E21B 49/08

(52) U.S. Cl. CPC E21B 47/005; E21B 49/02; E21B 49/08; E21B 2200/20

(57) ABSTRACT

A method is disclosed, where the method includes obtaining a water-leg sample from a carbonate facies and an oil-leg sample from the carbonate facies and determining a characteristic of a cement in each sample. The method further includes determining a volume of cement in each sample and determining a binary change flag indicating a temporal change in pore water geochemistry based, at least in part, on the volume and the characteristic of the cement in each sample.

### BACKGROUND

A depositional environment may refer to a specific area in which sediments may be deposited. FIG. 1, for example, shows a schematic (100) of a plurality of exemplary sedimentary depositional environments. Each type of sedimentary depositional environment may fall into a broader category, each of which may have distinct characteristics which infer different facts regarding the geological history of the specific environment.

For example, continental depositional environments may include depositional environments created by moving water over a body of land. Fluvial depositional environments (102) may refer to depositional environments created by streams. Common sediments in fluvial depositional environments may include gravel, sand and silt. Lacustrine depositional environments (104) may refer to depositional environments created by lakes, where common sediments include sand, silt, and clay. Aeolian depositional environments (106) typically occur in desert and are formed by wind activity. Alluvial depositional environments (108) refer to a specific type of fluvial deposit caused by moving water in a fan shape.

Transitional depositional environments refer to depositional environments created at the transition between a body of land and a body of water. Transitional depositional environments may include lagoonal areas (110), beach areas (112), deltaic areas (114), and tidal areas (116). Lagoonal areas (110) are characterized as a shallower body of water separated from a larger body of water by a thin land strip, where the thin land strip minimizes transportation of sediment. Common sediment in such an area includes carbonates.

Marine depositional environments refer to depositional environments created by moving bodies of water, specifically by waves, tidal currents, and ocean currents. Examples of marine depositional environments include shallow water marine environments (118), deep water marine environments (120), and reefs (122). In particular, carbonates may be formed in shallow water marine environments (120) formed in tropical locations and reefs (122). Glacial depositional environments (124) and evaporite depositional environments (126) may be categorized based on their mode of formation.

Cementation in carbonate reservoirs is a process that may alter rock porosity, permeability, and elastic properties, among other properties. Calcite and dolomite cementation is dominantly driven by the rock-fluid interaction whenever the water/rock ratio is relatively high. As such, both rock mineralogy and fluid chemistry have a significant impact on cement formation. Cementation in hydrocarbon-saturated pores is much slower than in brine-saturated pores.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a method, where the method may include obtaining a water-leg sample from a carbonate facies and an oil-leg sample from the carbonate facies and determining a characteristic of a cement in each sample. The method further includes determining a volume of cement in each sample and determining a binary change flag indicating a temporal change in pore water geochemistry based, at least in part, on the volume and the characteristic of the cement in each sample.

In another aspect, embodiments disclosed herein relate to a non-transitory computer-readable medium storing instructions. The instructions may include functionality for receiving a water-leg sample from a carbonate facies and an oil-leg sample from the carbonate facies, receiving a characteristic of a cement in each sample, and receiving a volume of cement in each sample. The instructions may also include functionality for determining a binary change flag indicating a temporal change in pore water geochemistry based, at least in part on the volume and the characteristic, of the cement in each sample.

In yet another aspect, embodiments disclosed herein relate to a system, which may include a sample tester. The sample tester may be configured to receive a water-leg sample from a carbonate facies and an oil-leg sample from the carbonate facies, determine a characteristic of a cement in each sample, and determine a volume of cement in each sample. The system may also include a computer processor configured to determine a binary change flag indicating a temporal change in pore water geochemistry based, at least in part on the volume and the characteristic, of the cement in each sample.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying FIGURES. Like elements in the various FIGURES are denoted by like reference numerals for consistency. The size and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn are not necessarily intended to convey any information regarding the actual shape of the particular elements and have been solely selected for ease of recognition in the drawing.

FIG. 1 shows a schematic of exemplary sedimentary depositional environments.

FIG. 2 shows a schematic of formation oil saturation in accordance with one or more embodiments.

FIGS. 3A-3C show schematics of cementation in a formation in accordance with one or more embodiments.

FIGS. 4A-4B show schematics of cementation in a formation in accordance with one or more embodiments.

FIG. 5 shows a flowchart of a decision tree in accordance with one or more embodiments.

FIG. 6 shows a flowchart of a method in accordance with one or more embodiments.

FIG. 7 shows a flowchart of a method in accordance with one or more embodiments.

FIG. 8 shows a computer system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

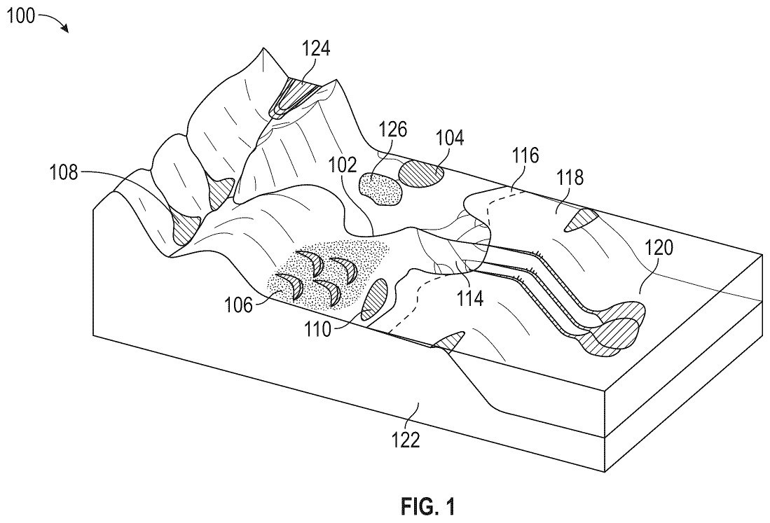

In the following description of FIGS. 1-8, any component described with regard to a FIGURE, in various embodiments disclosed herein, may be equivalent to one or more like-named components described with regard to any other FIGURE. For brevity, descriptions of these components will not be repeated with regard to each FIGURE. Thus, each and every embodiment of the components of each FIGURE is incorporated by reference and assumed to be optionally present within every other FIGURE having one or more like-named components. Additionally, in accordance with various embodiments disclosed herein, any description of the components of a FIGURE is to be interpreted as an optional embodiment which may be implemented in addition to, in conjunction with, or in place of the embodiments described with regard to a corresponding like-named component in any other FIGURE.

It is to be understood that the singular forms “a,” “an,” and “the” include plural referents unless the context clearly dictates otherwise. Thus, for example, reference to “a seismic data set” includes reference to one or more of such seismic data set.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

In one aspect, embodiments disclosed herein relate to a system and method of quantifying calcite or dolomite cementation in carbonate reservoirs where pore fluid type is associated and linked to different porosity and cement volume trends. The focus of embodiments disclosed herein is on differences of pore network between zones saturated with different pore fluids within the same reservoir facies such that the observed differences in pore network are not due to heterogeneity of reservoir facies. Specifically, one or more embodiments disclosed herein are based on a comparison of petrophysical properties between the hydrocarbon bearing interval and the brine bearing interval within the same reservoir facies. Once porosity is assessed along with the cement volume and fluid type, it is possible to estimate the amount of cement corresponding to both prior and post hydrocarbon stages. Furthermore, changes in water chemistry that resulted in forming different cement minerals may be deduced, which ultimately adds up toward a more comprehensive reservoir characterization.

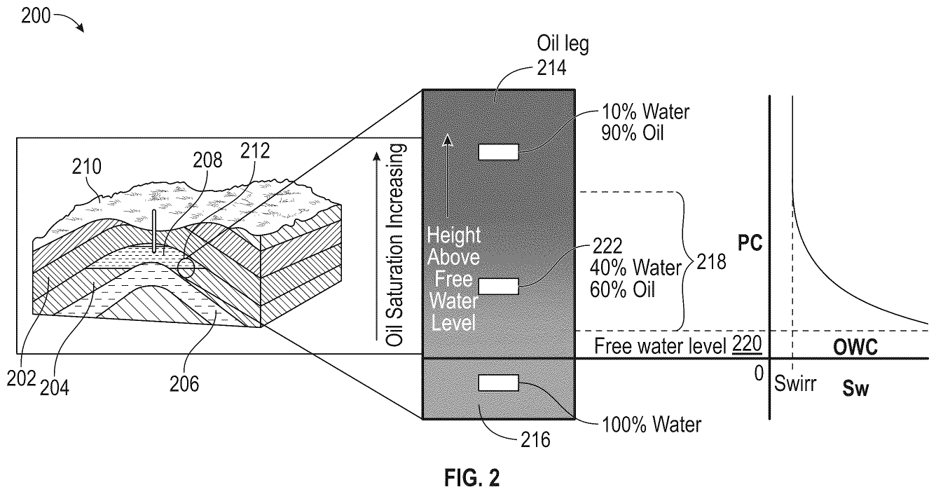

Turning now to FIG. 2, FIG. 2 depicts a schematic of oil saturation within a formation. A formation (200) may be formed by a variety of layers, including cap rock (202) and reservoir rock (204). Within the formation (200), there may also be layers of water (206), oil (208), and gas (210). In some formations (200), there may be a transition area (212), where oil (208) may transition into water (206). In such transition areas (212), there may be an oil leg (214) and a water leg (216) separated by a transition zone (218). A free water level (220) may exist in the water leg (216).

An oil leg (214) may refer to a location within the transition area (212) which is predominantly oil. Conversely, a water leg (216) may refer to a location within the transition area (212) which is predominantly water. The transition zone (218) extends between the oil leg (214) and the water leg (216) and refers to a location with a mixture of both oil and water. Areas in the transition zone (218) closer to the water leg (216) have a high proportion of water than oil, and areas closer to the oil leg (214) have a higher proportion of oil than water. For example, at one point (222) in the transition zone (218), the mixture may contain 40% water and 60% oil. Further, the greater the height above the free water level (220), the greater the proportion of oil in the mixture. An increased oil proportion may also be referred to as an increased oil saturation.

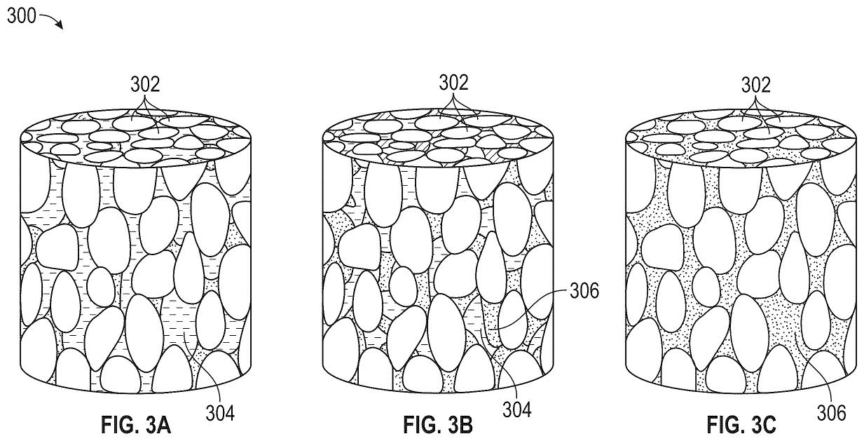

Turning now to FIGS. 3A-3C, FIGS. 3A-3C show various stages of cementation within a formation according to one or more embodiments. Cementation refers to a process of precipitation of cement between mineral or rock grains. For example, FIG. 3A shows a portion of a formation sample (300), which contains a plurality of rock grains (302). In formations such as formation sample (300), prior to cementation, the space between rock grains (302) may be filled by water (304). During cementation, some of the water (304) may be replaced with cement (306), as can be seen in FIG. 3B. FIG. 3C shows the formation (300) at the conclusion of the cementation process. As can be seen in FIG. 3C, no water is present in the cemented formation.

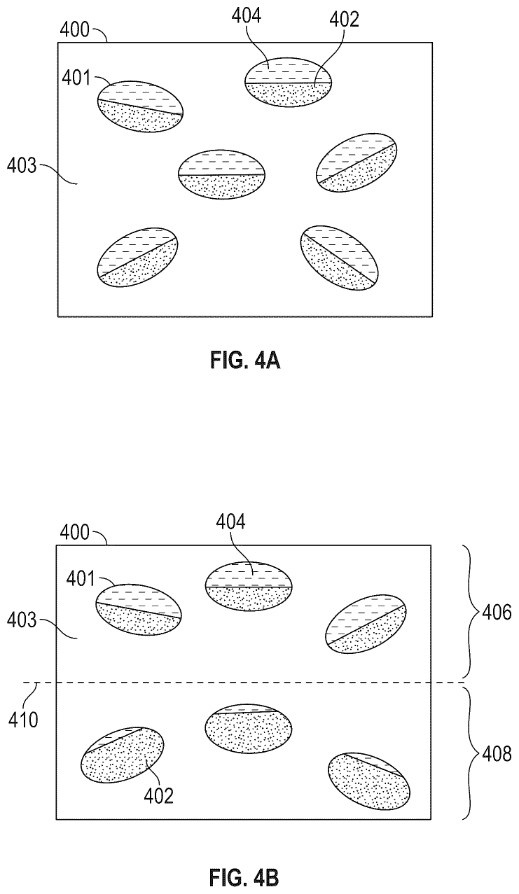

Turning now to FIGS. 4A-4B, 4A-4B show schematics of cementation in a formation in accordance with one or more embodiments. More specifically, FIG. 4A shows cementation in a formation pre-oil emplacement and FIG. 4B shows cementation in a formation post oil emplacement. Oil emplacement refers to the process by which oil is introduced into a formation. As shown in FIG. 4A, before oil emplacement, a carbonate reservoir facies (400) may include rock (403), which may have pores (401). The pores (401) may contain pore-filling cement particles (402). In one or more embodiments, each pore (401) may not be filled completely with pore-filling cement. Water (404) may fill the remaining space in each pore (401).

Following oil emplacement, as shown in FIG. 4B, the carbonate reservoir facies (400) may be split into an oil leg and a water leg. In one and more embodiments, the oil leg may also be referred to as the hydrocarbon bearing rock interval (406) and the water leg may also be referred to as the water bearing rock interval (408). The hydrocarbon bearing rock interval (406) and water bearing rock interval (408) may be separated by oil water contact (OWC) (410). In one or more embodiments, the pore-filling cement particles (402) in the hydrocarbon bearing rock interval (406) may account for early diagenesis and late diagenesis up until the time of oil emplacement. Further, the pore-filling cement particles (402) in the water bearing rock interval (408) may account for early diagenesis and late diagenesis. Diagenesis may refer to a physical, chemical, or biological alteration of sediments into sedimentary rocks at low temperatures and pressures. Diagenesis may result in changes to a rock's original mineralogy and texture.

The separation of the oil leg and the water leg may provide a variety of information regarding the rock and water geometry in each leg. For example, using methods and systems described herein, it may be possible to analyze the cement type and volume in each of the oil leg and the water leg, which may infer details regarding whether the water geochemistry altered after a hydrocarbon charge, where hydrocarbon charge may be another phrase for describing oil emplacement within a reservoir. After oil emplacement, diagenesis halts in pores containing hydrocarbons. In contrast, pores containing water in the same formation continue to be cemented further, as shown in FIG. 4B. Visually, this may appear as pores (401) in the water bearing rock interval (408) containing significantly more pore-filling cement than those in the hydrocarbon bearing rock interval (406). This results in a noticeable difference in porosity between the oil leg and the water leg.

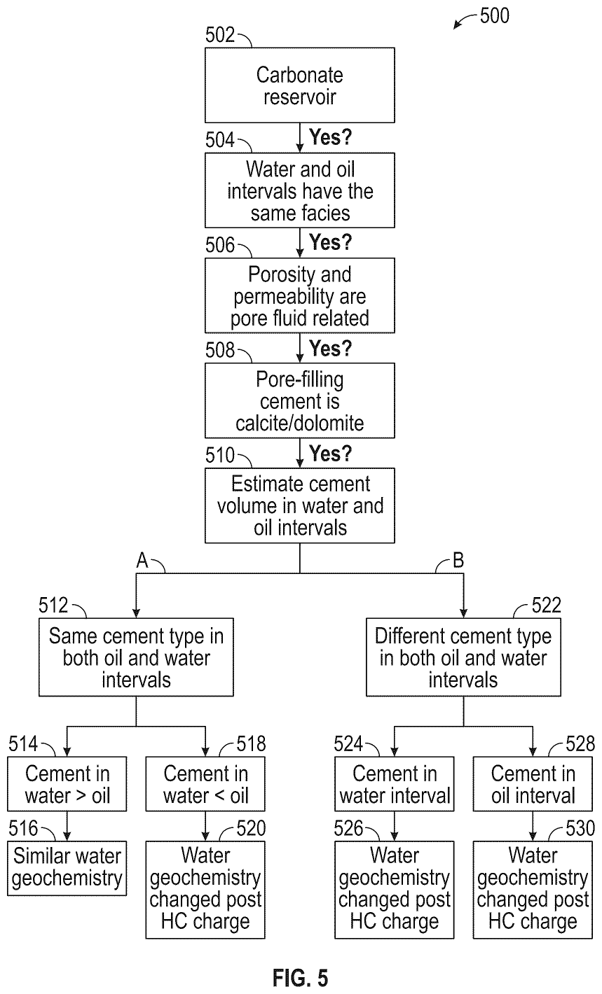

FIG. 5 depicts a flowchart in accordance with one or more embodiments. More specifically, FIG. 5 depicts a flowchart of a two-level decision tree (500) based on a determined cement volume and a determined cement characteristic. Further, one or more blocks in FIG. 5 may be performed by one or more components as described in FIGS. 1-4. While the various blocks in FIG. 5 are presented and described sequentially, one of ordinary skill in the art will appreciate that some or all of the blocks may be executed in different orders, may be combined, may be omitted, and some or all of the blocks may be executed in parallel. Furthermore, the blocks may be performed actively or passively.

In each of the block of decision tree (500), an operator may only progress from one block to another if the requirements of the first block are met. For example, block 502 requires that an obtained sample be composed of carbonate reservoir facies. If the sample is not composed of carbonate reservoir facies, the operator may not proceed to block 504 and must instead procure a new sample and begin the decision tree again. Likewise, if any of the conditions of the blocks later in the decision tree are not met, the operator must begin again from block 502 with a new sample.

In block 502, a sample may be obtained and may be analyzed to determine if the sample contains rock from a carbonate reservoir, where the sample contains both a water leg and an oil leg. Next, in block 504 the sample is analyzed to confirm that both the water leg and the oil leg have the same facies type. For example, in one or more embodiments, the facies type may be packstone, mudstone, or grainstone. In block 506, porosity and permeability of both the water leg and the oil leg are confirmed to be pore fluid related. In one or more embodiments, the porosity and permeability in the oil leg may be greater than those in the water leg. Analysis of porosity and permeability may be completed at borehole scale using petrophysical evaluation of wireline data. Alternatively, analysis of porosity and permeability may be completed using core description or conventional laboratory methods done on a sufficient volume of samples.

In block 508, the type of pore-filling cement may be confirmed, where type refers to the mineralogy of the cement. Suitable samples include pore-filling cement that is either calcite or dolomite in nature. In one or more embodiments, cement type may be identified using a scanning electron microscope. Next, as shown in block 510, cement volume in each of the water leg and the oil leg may be estimated. In one or more embodiments, blocks 508 and 510 may completed using one or more of the following analysis techniques: thin-section analysis, x-ray diffraction analysis, x-ray fluorescence analysis, and computed-tomography. Further, any other suitable analysis method may be utilized in place of the above-mentioned methods without departing from the scope disclosed herein.

If the same cement type is present in both the oil leg and the water leg, route A may be followed, as shown in block 512. In a sample with the same type of cement in each of the oil and water legs, there may be situations where the volume of cement in the water leg is larger than that in the oil leg, as shown in block 514, and there may also situations where the volume of cement in the water leg is less than that in the oil leg, as shown in block 518. Differences in cement volume may be linked to the rate of cementation and a change in water chemistry post hydrocarbon emplacement.

If the volume of cement in the water leg is greater than that in the oil leg, it may be concluded that both the oil and water legs may have similar water geochemistry, as shown in block 516. If the volume of cement in the water leg is less than that in the oil leg, it may be concluded that the water geochemistry changed post hydrocarbon charge, as shown in block 520.

If different cement types are present in the oil leg and the water leg, route B may be followed, as shown in block 522. There may be situations where cement is present in the water leg, as shown in block 524. There may also be situations in which cement is present in the oil leg, as shown in block 528. The presence of cement in only the water leg or only the oil leg indicates that water geochemistry changed post hydrocarbon charge, as shown in block 526 and 530.

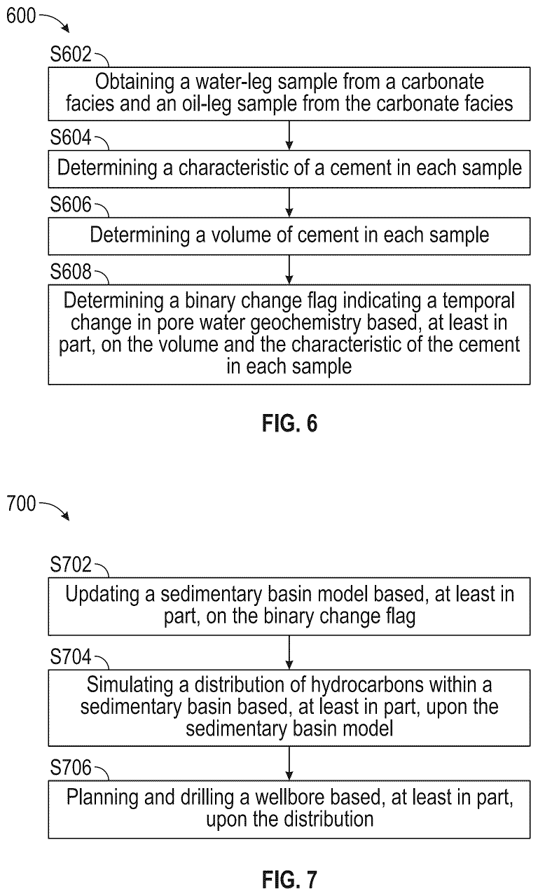

FIG. 6 depicts a flowchart in accordance with one or more embodiments. More specifically, FIG. 6 depicts a flowchart (600) of a method of calculating an average total organic carbon value corresponding to the net source rock thickness. Further, one or more blocks in FIG. 6 may be performed by one or more components as described in FIGS. 1-4. While the various blocks in FIG. 6 are presented and described sequentially, one of ordinary skill in the art will appreciate that some or all of the blocks may be executed in different orders, may be combined, may be omitted, and some or all of the blocks may be executed in parallel. Furthermore, the blocks may be performed actively or passively.

Initially, in step S602, a carbonate facies may be collected, and a water leg sample and an oil leg sample may be obtained therefrom. In one or more embodiments, the water leg sample and the oil leg sample may both be core samples. Next, as shown in step S604, a characteristic of a cement in each sample may be determined. In one or more embodiments, the characteristic may be mineral composition. In such embodiments, determining the mineral composition may include performing a fluorescence study of each sample.

In step S606, a volume of cement in each sample may be determined. In one or more embodiments, determining the volume of cement may include cutting a thin section from each sample and performing a scanning electronic microgram of the thin section. However, there may be other methods of determining cement volume, all of which fall under the scope of this disclosure.

In step S608, a binary change flag may be determined, where the binary change flag indicates a temporal change in pore water geochemistry based, at least in part, on the volume and the characteristic of the cement in each sample. In one or more embodiments, a binary change flag may be, for example, a Boolean value such as a ‘yes’ or ‘no’. In one or more embodiments, determining a binary change flag may include applying a two-level decision tree, such as decision tree (500). In some embodiments, the first level may compare the volume of each sample, and the second level may compare the characteristic of each sample.

In other embodiments, determining a binary change flag may include assigning an affirmative value to a binary characteristic flag when the characteristic of each sample is essential similar, and assigning an affirmative value to a binary volume flag when the volume of cement in the water leg sample is greater than the volume of cement in the oil leg sample. For example, in situations where the characteristic of each sample is essentially similar, the binary characteristic flag may be ‘yes’. In the same manner, in situations where the volume of each sample is essentially similar, the binary volume flag may be ‘yes’. In such embodiments, an affirmative value may be assigned to the binary change flag when the value of the binary characteristic flag is affirmative and the value of the binary volume flag is affirmative. The binary change flag may be ‘yes’ if both the binary characteristic flag and the binary volume flag are also ‘yes’. In one or more embodiments, characteristics which are essentially similar may include samples with the same mineral type and a similar texture. In one or more embodiments, volumes which are essentially similar may include volumes which are within a certain degree of each other. For example, in some embodiments, essentially similar may indicate volumes which are different by no more than 5%.

In one or more embodiments, an affirmative binary change flag may indicate similar water geochemistry in each of the oil leg sample and the water leg sample. In decision tree (500), for example, an affirmative binary change flag may be produced in block 616. In the same manner, a non-affirmative binary change flag may indicate a change in pore water geochemistry following oil emplacement in the reservoir. In decision tree (500), for example, a non-affirmative binary change flag may be produced in blocks 520, 526, and 530.

In one or more embodiments, the method described in flowchart (600) may be executed using a sample tester system and a computer processor. For example, the sample tester may receive the water and oil leg samples, and determine the characteristic and volume of cement in each sample. The computer processor may the determine the binary change flag based on the determined cement characteristic and volume. The sample tester may include a variety of equipment necessary for performing the characteristic and volume of cement in each sample.

FIG. 7 depicts a flowchart in accordance with one or more embodiments. More specifically, FIG. 7 depicts a flowchart (700) of a method of calculating an average total organic carbon value corresponding to the net source rock thickness. Further, one or more blocks in FIG. 7 may be performed by one or more components as described in FIGS. 1-4. While the various blocks in FIG. 7 are presented and described sequentially, one of ordinary skill in the art will appreciate that some or all of the blocks may be executed in different orders, may be combined, may be omitted, and some or all of the blocks may be executed in parallel. Furthermore, the blocks may be performed actively or passively.

In one or more embodiments, the method described in flowchart (700) may be performed immediately following the method shown in flowchart (600), using the determined binary change flag. In step S702, a sedimentary basin model may be updated based, at least in part, on the binary change flag. In one or more embodiments, sedimentary basin models may be developed to better understand the geological history of a formation and to inform predictions regarding future formation production. Sedimentary basin models may also be used to quantify porosity reduction and cementation linked to diagenesis events throughout the geological history of the formation.

In step S704, a distribution of hydrocarbons within a sedimentary basin may be simulated based, at least in part, on the sedimentary basin model. Further, as shown in step S706, planning and drilling of a wellbore may be completed based, at least in part, on the hydrocarbon distribution. In one or more embodiments, an area of greater hydrocarbon quantity, as indicated by the hydrocarbon distribution, may represent a potential wellbore drilling location. As such, planning a wellbore may include isolating areas of greater hydrocarbon quantity from the hydrocarbon distribution.

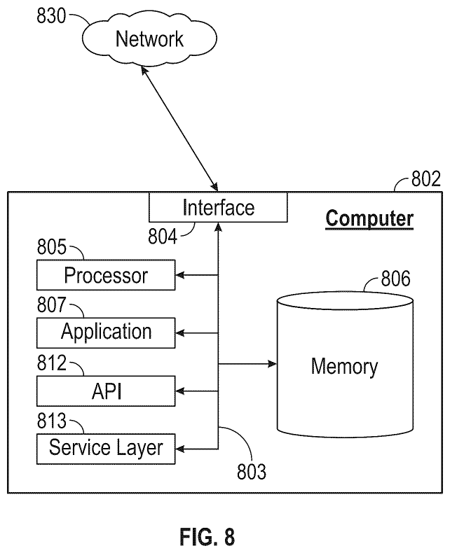

FIG. 8 depicts a block diagram of a computer system (802) used to provide computational functionalities associated with described algorithms, methods, functions, processes, flows, and procedures as described in this disclosure, according to one or more embodiments. For example, the computer system (802), and the processor of the computer system, may be used to perform one or more steps of the flowchart (calculations, determinations, etc.) in FIGS. 6-7. The illustrated computer (802) is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (802) may include a computer that includes an input device, such as a keypad, keyboard, touch screen, or other device that can accept user information, and an output device that conveys information associated with the operation of the computer (802), including digital data, visual, or audio information (or a combination of information), or a GUI.

The computer (802) can serve in a role as a client, network component, a server, a database or other persistency, or any other component (or a combination of roles) of a computer system for performing the subject matter described in the instant disclosure. The illustrated computer (802) is communicably coupled with a network (830). In some implementations, one or more components of the computer (802) may be configured to operate within environments, including cloud-computing-based, local, global, or other environment (or a combination of environments).

At a high level, the computer (802) is an electronic computing device operable to receive, transmit, process, store, or manage data and information associated with the described subject matter. According to some implementations, the computer (802) may also include or be communicably coupled with an application server, e-mail server, web server, caching server, streaming data server, business intelligence (BI) server, or other server (or a combination of servers).

The computer (802) can receive requests over network (830) from a client application (for example, executing on another computer (802)) and responding to the received requests by processing the said requests in an appropriate software application. In addition, requests may also be sent to the computer (802) from internal users (for example, from a command console or by other appropriate access method), external or third-parties, other automated applications, as well as any other appropriate entities, individuals, systems, or computers.

Each of the components of the computer (802) can communicate using a system bus (803). In some implementations, any or all of the components of the computer (802), both hardware or software (or a combination of hardware and software), may interface with each other or the interface (804) (or a combination of both) over the system bus (803) using an application programming interface (API) (812) or a service layer (813) (or a combination of the API (812) and service layer (813). The API (812) may include specifications for routines, data structures, and object classes. The API (812) may be either computer-language independent or dependent and refer to a complete interface, a single function, or even a set of APIs. The service layer (813) provides software services to the computer (802) or other components (whether or not illustrated) that are communicably coupled to the computer (802). The functionality of the computer (802) may be accessible for all service consumers using this service layer. Software services, such as those provided by the service layer (813), provide reusable, defined business functionalities through a defined interface. For example, the interface may be software written in JAVA, C++, or other suitable language providing data in extensible markup language (XML) format or another suitable format. While illustrated as an integrated component of the computer (802), alternative implementations may illustrate the API (812) or the service layer (813) as stand-alone components in relation to other components of the computer (802) or other components (whether or not illustrated) that are communicably coupled to the computer (802). Moreover, any or all parts of the API (812) or the service layer (813) may be implemented as child or sub-modules of another software module, enterprise application, or hardware module without departing from the scope of this disclosure.

The computer (802) includes an interface (804). Although illustrated as a single interface (804) in FIG. 8, two or more interfaces (804) may be used according to particular needs, desires, or particular implementations of the computer (802). The interface (804) is used by the computer (802) for communicating with other systems in a distributed environment that are connected to the network (830). Generally, the interface (804) includes logic encoded in software or hardware (or a combination of software and hardware) and operable to communicate with the network (830). More specifically, the interface (804) may include software supporting one or more communication protocols associated with communications such that the network (830) or interface's hardware is operable to communicate physical signals within and outside of the illustrated computer (802).

The computer (802) includes at least one computer processor (805). Although illustrated as a single computer processor (805) in FIG. 8, two or more processors may be used according to particular needs, desires, or particular implementations of the computer (802). Generally, the computer processor (805) executes instructions and manipulates data to perform the operations of the computer (802) and any machine learning networks, algorithms, methods, functions, processes, flows, and procedures as described in the instant disclosure.

The computer (802) also includes a memory (806) that holds data for the computer (802) or other components (or a combination of both) that can be connected to the network (830). For example, memory (806) can be a database storing data consistent with this disclosure. Although illustrated as a single memory (806) in FIG. 8, two or more memories may be used according to particular needs, desires, or particular implementations of the computer (802) and the described functionality. While memory (806) is illustrated as an integral component of the computer (802), in alternative implementations, memory (806) can be external to the computer (802).

The application (807) is an algorithmic software engine providing functionality according to particular needs, desires, or particular implementations of the computer (802), particularly with respect to functionality described in this disclosure. For example, application (807) can serve as one or more components, modules, applications, etc. Further, although illustrated as a single application (807), the application (807) may be implemented as multiple applications (807) on the computer (802). In addition, although illustrated as integral to the computer (802), in alternative implementations, the application (807) can be external to the computer (802).

There may be any number of computers (802) associated with, or external to, a computer system containing a computer (802), wherein each computer (802) communicates over network (830). Further, the term “client,” “user,” and other appropriate terminology may be used interchangeably as appropriate without departing from the scope of this disclosure. Moreover, this disclosure contemplates that many users may use one computer (802), or that one user may use multiple computers (802).

Embodiments of the present disclosure may provide at least one of the following advantages. By analyzing cement mineralogy, the presence of different cement types in each of the oil and water legs can provide information regarding cementation rate pre- and post-hydrocarbon emplacement. For example, if the hydrocarbon charge time is known, average cementation rate after the hydrocarbon charge can be calculated as cement volume divided by time. A similar calculation may be performed for average cementation rate before the hydrocarbon charge. In one or more embodiments, the hydrocarbon charge may occur over a period of time. Cement mineralogy analysis can also provide information regarding paleo water chemistry.

The methods and systems disclosed herein can also be used to assist in constructing a rock physics characterization of a reservoir. For example, accounting for the difference in cement volume when constructing rock physics templates for each of the oil and water legs can better inform the prediction of associated elastic properties and interpretation of seismic signatures. In particular, the characterization results of the methods disclosed herein may be implemented in seismic interpretation workflows to predict fluid type and porosity for reservoir characterization analyses. Predictions of cement variations over time can also provide additional constraints for numerical diagenetic modelling, improving the accuracy of the generated models.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112(f) for any limitations of any of the claims herein, except for those in which the claim expressly uses the words ‘means for’ together with an associated function.

What is claimed:

1. A method, comprising:
obtaining a water-leg sample from a carbonate facies and an oil-leg sample from the carbonate facies;
determining a characteristic of a cement in each sample;
determining a volume of cement in each sample; and
determining a binary change flag indicating a temporal change in pore water geochemistry based, at least in part, on the volume and the characteristic of the cement in each sample.

2. The method of claim 1, further comprising:
updating a sedimentary basin model based, at least in part, on the binary change flag;
simulating a distribution of hydrocarbons within a sedimentary basin based, at least in part, upon the sedimentary basin model; and
planning and drilling a wellbore based, at least in part, upon the distribution.

3. The method of claim 1, wherein the characteristic comprises mineral composition.

4. The method of claim 3, wherein determining the mineral composition comprises performing a fluorescence study of each sample.

5. The method of claim 1, wherein the two samples comprise two core samples.

6. The method of claim 1, wherein determining the volume comprises:
cutting a thin section from each sample; and
determining the volume of cement using a scanning electronic microgram of the thin section.

7. The method of claim 1, wherein determining a binary change flag comprises applying a two-level decision tree, wherein a first level compares the characteristic of each sample and a second level compares the volume of each sample.

8. The method of claim 1, wherein determining a binary change flag, comprises:
assigning an affirmative value to a binary characteristic flag when the characteristic of each sample is essential similar;
assigning an affirmative value to a binary volume flag when the volume of cement in the water-leg sample is greater than the volume of cement in the oil-leg sample; and
assigning an affirmative value to the binary change flag when the value of the binary characteristic flag is affirmative and the value of the binary volume flag is affirmative.

9. A non-transitory computer-readable medium storing instructions, the instructions comprising functionality for:
receiving a water-leg sample from a carbonate facies and an oil-leg sample from the carbonate facies;
receiving a characteristic of a cement in each sample;
receiving a volume of cement in each sample; and
determining a binary change flag indicating a temporal change in pore water geochemistry based, at least in part on the volume and the characteristic, of the cement in each sample.

10. The non-transitory computer-readable medium of claim 9, further comprising:
updating a sedimentary basin model based, at least in part, on the binary change flag;
simulating a distribution of hydrocarbons within a sedimentary basin based, at least in part, upon the sedimentary basin model; and
planning a wellbore drilling operation based, at least in part, upon the distribution.

11. The non-transitory computer-readable medium of claim 9, wherein determining a binary change flag, comprises:
assigning an affirmative value to a binary characteristic flag when the characteristic of each sample is essential similar;
assigning an affirmative value to a binary volume flag when the volume of cement in the water-leg sample is greater than the volume of cement in the oil-leg sample; and
assigning an affirmative value to the binary change flag when the value of the binary characteristic flag is affirmative and the value of the binary volume flag is affirmative.

12. The non-transitory computer-readable medium of claim 9, wherein determining a binary change flag comprises applying a two-level decision tree, wherein a first level compares the characteristic of each sample and a second level compares the volume of each sample.

13. A system, comprising:
a sample tester, configured to:
receive a water-leg sample from a carbonate facies and an oil-leg sample from the carbonate facies;
determine a characteristic of a cement in each sample; and
determine a volume of cement in each sample; and
a computer processor, configured to:
determine a binary change flag indicating a temporal change in pore water geochemistry based, at least in part on the volume and the characteristic, of the cement in each sample.

14. The system of claim 13, wherein the computer processor is further configured to:
assign an affirmative value to a binary characteristic flag when the characteristic of each sample is essential similar;
assign an affirmative value to a binary volume flag when the volume of cement in the water-leg sample is greater than the volume of cement in the oil-leg sample; and
assign an affirmative value to the binary change flag when the value of the binary characteristic flag is affirmative and the value of the binary volume flag is affirmative.

15. The system of claim 13, wherein the computer processor is further configured to:
update a sedimentary basin model based, at least in part, on the binary change flag;
simulate a distribution of hydrocarbons within a sedimentary basin based, at least in part, upon the sedimentary basin model; and
plan and drill a wellbore based, at least in part, upon the distribution.

16. The system of claim 13, wherein the characteristic comprises mineral composition.

17. The system of claim 16, wherein the sample tester is further configured to perform a fluorescence study of each sample.

18. The system of claim 13, wherein the computer processor is further configured to apply a two-level decision tree, wherein a first level compares the characteristic of each sample and a second level compares the volume of each sample.

19. The system of claim 13, wherein the sample tester is further configured to:
cut a thin section from each sample; and
determine the volume of cement using a scanning electronic microgram of the thin section.

20. The system of claim 13, wherein the two samples comprise two core samples.
